(19) United States
(12) Patent Application Publication
(10) Pub. No.: US 2005/0181508 A1
(43) Pub. Date: Aug. 18, 2005

(54) Device for multiple simulataneous gene transfer

(76) Inventors: Sarah Fredriksson, Staffanstorp (SE); Dario Kriz, Hoor (SE)

Correspondence Address: BURNS DOANE SWECKER & MATHIS L L P, POST OFFICE BOX 1404, ALEXANDRIA, VA, 22313-1404, US

(21) Appl. No.: 10/515,418

(22) PCT Filed: Jun. 6, 2003
(86) PCT No.: PCT/SE03/00938

(30) Foreign Application Priority Data
Jun. 7, 2002 (SE) 02017630

(51) Int. Cl. C12N 15/87

(52) U.S. Cl. 435/459; 435/173.5; 435/285.1

(57) ABSTRACT

The present invention relates to a device for membrane passage, which comprises at least two magnetic fields generating means, each of which can generate an alternating magnetic field in a spatially limited area located in or in the immediate vicinity of the means, and a separate sample containing membrane-enveloped biological material in each spatially limited area, the device being further connected to a computer program which controls the magnetic field generating means with respect to point of time and duration for activating each individual means.

### FIELD OF THE INVENTION

The present invention relates to equipment for insertion of molecular units in membrane-enveloped biological material in multiple samples by means of a magnetic alternating field and a computer program, methods for insertion of molecular units in membrane-enveloped biological material in multiple samples by means of a magnetic alternating field and a computer program and uses thereof.

### BACKGROUND ART

Magnetism and magnetically susceptible particles have been used for a long time in various biochemical and medical applications, ref. 1. When paramagnetic materials are exposed to an external alternating homogeneous magnetic field, heat and motion are generated. This generation of heat/motion, in particular in combination with superparamagnetic nanoparticles, is used, inter alia, for transfection of cells, ref. 2. The present invention comprises a subcomponent which is based on a device as described in ref. 2.

To allow a comparative study of various samples in one or more different magnetic fields where all samples must be treated in exactly the same way, it is necessary for the transfection of all samples to be carried out simultaneously. With the devices that are currently available, this is not possible since each sample in the series must be mixed and treated individually. Then, on the one hand the cell suspension has to be incubated on ice, in which case the cell suspension cannot be assumed to be exactly the same in all samples, and on the other hand it is difficult to treat so many samples in exactly the same way.

By means of the present invention, based on a construction comprising at least two coils which can be supplied with current simultaneously or sequentially and which can be controlled individually by a computer program or software, this need is satisfied. The advantages of the present invention also include that it is adjusted to standard multisample containers, so-called microtiter plates, comprising for instance 48 or 96 wells, and that it can easily be equipped with a robotic sample handling system or be included as part of existing automated systems where the transfection of cells constitutes part of a longer sequence of measures. A further advantage is that the transfection can be made more efficient in terms of time since multiple samples can be treated in parallel.

A device for increasing the thermal and/or kinetic energy of magnetically susceptible particles is previously known, said device comprising at least two magnetic field generating means, of which at least one is a coil, between which means an alternating magnetic gradient field can be generated in a spatially limited area, in which human or animal tissue can be inserted, said alternating magnetic gradient field causing in an increase of the thermal and/or kinetic energy of the magnetically susceptible particles which have been supplied to said tissue, the increased thermal and/or kinetic energy of the magnetically susceptible particles selectively reducing, deactivating or destroying endogenic or exogenic biological structures in said tissue, ref. 3. In the present invention, the coils are not used to generate an alternating gradient field in a spatially limited area, but to generate homogeneous alternating magnetic fields in two or more spatially limited areas Moreover the coils are controlled by a computer program, individually or simultaneously.

A device based on a plurality of coils for stimulation of respiratory muscles, ref. 4, and a device for treatment of cancer, ref. 5, are previously known. These devices are designed for medical therapy and therefore cannot be used in simultaneous gene transfer in multiple samples.

The invention can be used, not only for transfection, but also for membrane passage in different types of gene transfers and molecule transfers (exemplified by DNA, RNA, genes, protein, peptides, antibodies, synthetic molecules and viruses) in the membrane-enveloped biological material (exemplified by cells, cell components, liposomes and viruses). Synthetic molecules for insertion in membrane-enveloped structures can be exemplified by fluorescent molecules and colouring matters. A conceivable application of the invention according to the invention thus is insertion of synthetic molecules which result in easy search of the coloured or fluorescent biological material.

### SUMMARY OF THE INVENTION

According to the present invention, a device is provided, which solves the problems in simultaneous treatment of multiple samples in gene transfers, such as transfection processes. Thus, a multisample handling device is provided, which can generate homogeneous alternating magnetic fields in two or more spatially limited areas. Each individual coil can be controlled and checked separately by a computer program. In the spatially defined areas, samples containing suspensions are inserted, which may contain cells, viruses, plasmids, DNA, RNA and magnetically susceptible particles. The increased thermal and/or kinetic energy of the magnetically susceptible particles causes transport of the molecular units, such as DNA, RNA, protein, peptides, into the cells or virus particles.

In one embodiment of the invention, a device for membrane passage is provided, which contains at least two magnetic field generating means, each of which can generate an alternating magnetic field in a spatially limited area located in or in the immediate vicinity of said means, and a separate sample containing membrane-enveloped biological material and magnetically susceptible particles can be inserted in each spatially limited area, said device being further connected to a computer program which controls the magnetic field generating means with respect to point of time and duration for activating each individual means. By the expression “activating” as used herein is thus meant the control, performed by the program, of the strength of the applied magnetic field, its frequency and of how long it is to be generated. It is a great advantage that each individual means can be controlled individually or simultaneously.

In another embodiment of the invention, a method is provided for insertion of molecular units in multiple samples containing membrane-enveloped biological material and magnetically susceptible particles simultaneously or sequentially, in which
a) each sample is inserted in a spatially limited area located in or in the vicinity of a magnetic field generating means;
b) the magnetic field generating means generates an alternating magnetic field by a computer program which controls the magnetic field generating means with respect to point of time and duration for activating each individual means,
c) the molecular units are inserted in the membrane-enveloped biological material through the pores that are produced by the generated alternating magnetic field.

In the present invention, the molecular units are selected among DNA, RNA, genes, proteins, antibodies and peptides, and the membrane-enveloped biological material is selected among stem cells, mammalian cells, malignant cells, plant cells, nerve cells, bacteria, viruses, cellular organelles, cell-membrane-enveloped structures, cell-wall-enveloped structures, liposomes, protozoa, parasites and combinations thereof.

In one embodiment of the invention, the device comprises at least two coils which are supplied with two independent alternating currents of the same frequency and amplitude.

Moreover the device can suitably be equipped with a thermostat for accurate control of the temperature of said samples and/or with a variable timing for accurate control of the time during which said samples are exposed to the alternating magnetic field.

In an embodiment of the device, the alternating magnetic field shifts with a frequency of 1 MHz and the field strength in said coils amounts to 100 Örstedt.

Cells/Cell components/Liposomes/Viruses to be treated may consist of bacteria, protoplasts, plant cells, mitochondria, viruses, protozoa, animal cells, mammalian cells and stem cells.

The magnetically susceptible particles suitably comprise a core of a metal oxide and an envelope containing Concanavalin A, other lectins, cell-binding proteins, cell-binding peptide sequences, antibodies or parts thereof and having a size of 0.1-300 nm.

### BRIEF DESCRIPTION OF THE DRAWINGS

FIG. 1 is a schematic illustration of the construction of an embodiment of the device according to the invention for generating 16 multiple alternating magnetic fields.

FIG. 2 is an illustration of an electronic circuit that can be used to supply the coils in one embodiment of the device according to the invention with an alternating current.

FIG. 3 is an illustration of a connection of each individual coil element, based on an oscillating circuit.

### DETAILED DESCRIPTION OF THE INVENTION

According to the present invention, a new device for use in multiple gene transfer is provided.

The invention will now be described in more detail by means of the drawings which illustrate embodiments of the invention.

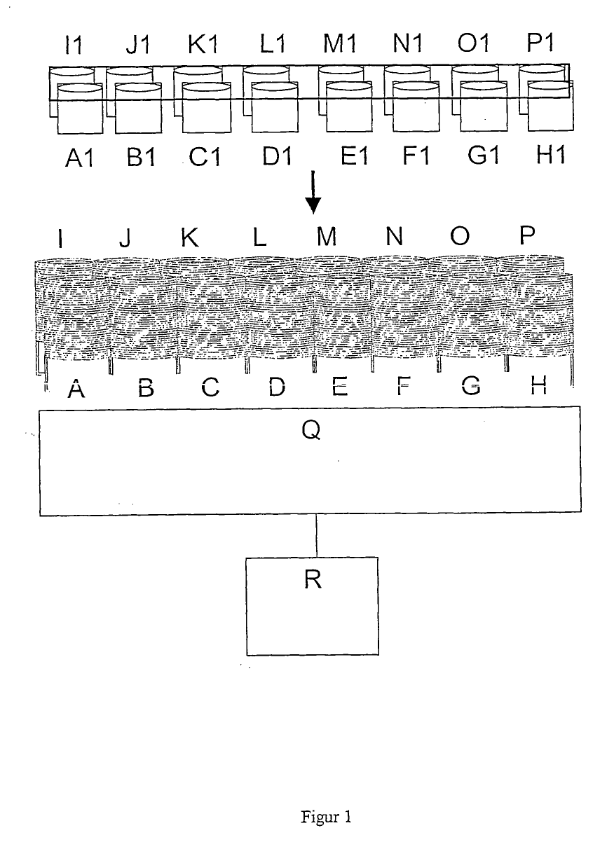

In order to generate multiple alternating magnetic fields, a device according to the invention is required, for instance as illustrated in FIG. 1. The functional principle is based on, for instance, 16 coils marked A-P being arranged in two juxtaposed rows. A control unit Q, by software in the form of a computer program, directs the current through the coils so that each coil can be supplied with current individually and independently of the other coils. This current supply, whose frequency and amplitude are controlled by the oscillator R, makes it possible for the coils to generate an alternating magnetic field in a predetermined sequence. In the coils, or in the their immediate vicinity (i.e. within a distance of 5 cm), a sample container containing 16 samples marked A1-P1 is arranged. Gene transfer in the samples is obtained after exposing the samples to the alternating magnetic field in the coils.

The present invention also comprises variants in which, for instance, current intensity, current control by software, number of coils, design of the coils and temperature control may be varied.

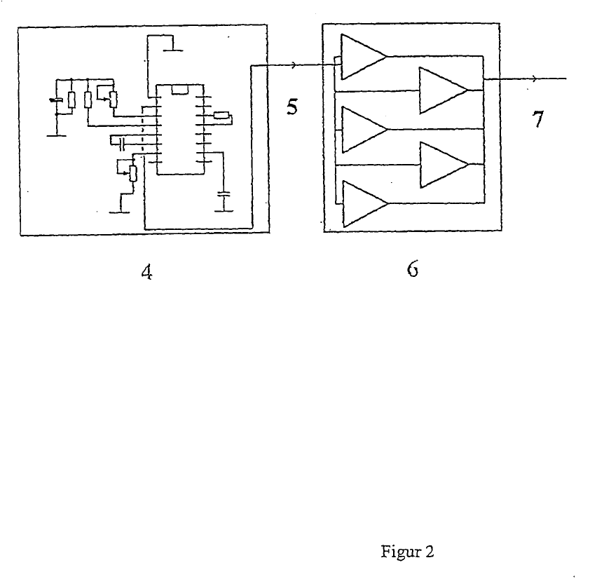

FIG. 2 illustrates an example of an electronic circuit that may be used to supply the coils in the device according to the invention with an alternating current. The circuit comprises an oscillator 4 based on the circuit XR2206, whose output signal 5 is amplified by a power amplifier step 6 connected in parallel and based on 5 circuits of the type PBD 3548/l (made by Ericsson), whose output signal 7 can drive an alternating current (max 1 MHz, 10 A) through one or more coils.

It is obvious to a person skilled in the art that the electronic circuit described above in FIG. 2 can easily be modified and that the same result can be obtained by various alternative prior-art connections of oscillators and power amplifiers. Such alternative connections are within the scope of the present invention.

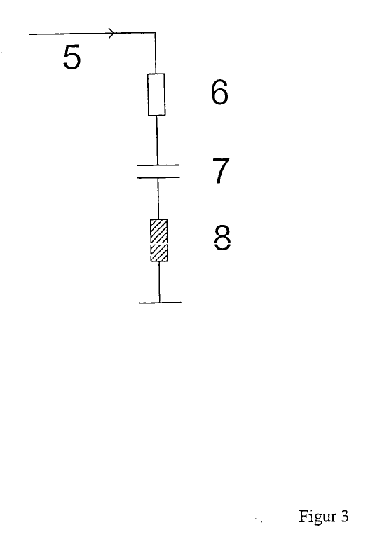

An example of the connection of the coils implies that each coil constitutes part of an oscillating circuit consisting of a 0.5 Ω resistance, a 127 pF capacitor and a 200 μH coil, connected in series, said oscillating circuit being supplied with alternating current as shown in FIG. 3.

Each coil in FIG. 1 constitutes part of an oscillating circuit according to FIG. 3 which, in turn, is connected to an oscillator and a drive circuit according FIG. 2. This means that there is a total of 16 sets of each component. In an alternative embodiment, 16 relays are used, which can independently connect the respective coils (and their oscillating circuit) to a single oscillator and drive circuit according to FIG. 2. The relays are controlled by software or a logically based system.

It is obvious to a person skilled in the art that the example described above can easily be modified and that the same result can be obtained by various alternative connections and coils.

### EXAMPLES

### Example 1

### Comparative Study of the Effect of Different Cell-Binding Epitopes on Transformation of E. coli with pUC18

A colony of E. coli BL121 was grafted from a minimum media plate to 5 ml culture medium and was then placed in a shaking incubator over night. The next day 0.4 ml of the overnight culture was grafted to 40 ml of new culture medium. The culture flask was again placed in the shaking incubator and the growth rate was controlled by withdrawing samples that were analysed with regard to absorbance at 600 nm. At the absorbance 600 nm=0.4 the cultivation was interrupted. The cells were centrifuged at 5000 g for 10 min. The cell pellets were washed with 40 ml 0.05 M CaCl₂, 1 mM MnCl₂, 0.15 m NaCl. The cells were centrifuged for 10 m at 5000 g and the cell pellets were resuspended in 4 ml 0.05 M CaCl₂, 1 mM MnCl₂.

The following was added to a microtiter plate with 48 sample wells:

Wells 1-8: Plasmid pUC18 (30 μg/ml) as Follows

well1=0 μl
well2=0.1 μl
well3=0.5 μl
well4=0.75 μl
well5=1.0 μl
well6=1.5 μl

10 μl cell suspension was added and the samples were incubated* for 10 min at room temperature in a shaking incubator, then 0.05 M CaCl₂, 1 mM MnCl₂ was added so that each sample had a final volume of 200 μl.
*Incubation does not take place until all wells have been filled with the respective samples.

Wells 7-16: 1 μl pUC18 (30 μg/ml) as distributed in wells 1-6 above, 10 μl cell suspension and the samples were incubated* for 10 min at room temperature in a shaking incubator. Then 20 μl Con-A ferrofluid ((μᵣ=1.00200), protein concentration: 0.01 mg/ml) was added.
*Incubation does not take place until all wells have been filled with the respective samples.

0.05 M CaCl₂, 1 mM MnCl₂ was added so that each sample had a final volume of 200 μl.

Wells 17-24: 1 μl pUC18 (30 μg/ml) as distributed in wells 1-6 above, 10 μl cell suspension and the samples were incubated* for 10 min at room temperature in a shaking incubator. Then 50 μl Con-A ferrofluid ((μᵣ=1.00200), protein concentration: 0.01 mg/ml) was added.
*Incubation does not take place until all wells have been filled with the respective samples.

0.05 M CaCl₂, 1 mM MnCl₂ was added so that each sample had a final volume of 200 μl.

Wells 25-32: 1 μl pUC18 (30 μg/ml) as distributed in wells 1-6 above, 10 μl cell suspension and the samples were incubated* for 10 min at room temperature in a shaking incubator. Then 20 μl antiOmpA ferrofluid ((μᵣ=1.00200), protein concentration: 0.01 mg/ml) was added.
*Incubation does not take place until all wells have been filled with the respective samples.

0.05 M CaCl₂, 1 mM MnCl₂ was added so that each sample had a final volume of 200 μl.

Wells 33-40: 1 μl pUC18 (30 μg/ml) as distributed in wells 1-6 above, 10 μl cell suspension and the samples were incubated* for 10 min at room temperature in a shaking incubator. Then 50 μl antiOmpA ferrofluid ((μᵣ=1.00200), protein concentration: 0.01 mg/ml) was added.
*Incubation does not take place until all wells have been filled with the respective samples.

0.05 M CaCl₂, 1 mM MnCl₂ was added so that each sample had a final volume of 200 μl.

Wells 41-48: 1 μl pUC18 (30 μg/ml) as distributed in wells 1-6 above, 10 μl cell suspension and the samples were incubated* for 10 min at room temperature in a shaking incubator. Then 50 μl COO⁻ ferrofluid (μᵣ=1.00200) was added.
*Incubation does not take place until all wells have been filled with the respective samples.

0.05 M CaCl₂, 1 mM MnCl₂ was added so that each sample had a final volume of 200 μl.

The microtiter plate was incubated for another 15 min at room temperature, after which the entire plate was put down in the device described in this application and exposed to a magnetic field 1 Mhz, 100 Oe for 20 s.

200 μm culture medium was added by a pipette to each sample, and then the plate was arranged for incubation at 37° C. for 45 min.

Samples of 100 μl were spread on agar plates containing IPTG, ampicillin and X-gal. The plates were incubated over night at 37° C., after which the number of blue colonies (positive transformants) was counted for each individual plate.

The results from this experiment were used to verify the effect of the different ferrofluids on the transfection at a varying content of DNA plasmid. This study of such a large number of samples cannot be carried out if each sample in the series were to be mixed and treated individually since on the one hand the cell suspension has to be incubated on ice, in which case the cell suspension cannot be assumed to be exactly the same in all samples and, on the other hand, it is difficult to treat so many samples in exactly the same way. By multisample treatment, it is further possible to obtain much more basic data in a considerably shorter time for different transfection studies, which makes it easier to guarantee the results.

### REFERENCES

1. Jordan A., Wust P., Scholz R., Faehling H., Krause J. & Felix R. Magnet Fluid Hyperthermia, 569-597, in Scientific and Clinical Applications of Magnetic Carriers, edited by Häfeli U., Schutt W., Teller J. and Zborowski M. Plenum Press 1997.
2. Fredriksson S., Kriz D., Sep. 8, 1999, WO 01/18168.
3. Fredriksson S., Kriz D., Sep. 8, 1999, WO 01/17611.
4. Mecchlenburg D., Oct. 17, 1997, WO 99/20339.
5. Gordon R. T., Sep. 23, 1983, U.S. Pat. No. 4,662,359.

1. A device for membrane passage comprising at least two magnetic field generating means, each of which can generate an alternating magnetic field in a spatially limited area located in or in the immediate vicinity of said means, and a separate sample containing membrane-enveloped biological material and magnetically susceptible particles can be inserted into each spatially limited area, said device being further connected to a computer program which controls the magnetic field generating means with respect to point of time and duration for activating each individual means.

2. A device as claimed in. claim 1, wherein the magnetic field generating means are coils and can consist of an electric conductor which is wound on a core consisting of air or a polymer or a substance having a relative magnetic permeability greater than 2.

3. A device as claimed in claim 1, wherein the magnetic field generating means are supplied with alternating currents of the same frequency and amplitude, and that said means are simultaneously supplied with said current.

4. A device as claimed in claim 1, wherein said device is equipped with a thermostat for accurate control of the temperature of said samples and/or that it is equipped with a variable timing for accurate control of the time during which said samples are exposed to the alternating magnetic field.

5. A device as claimed in claim 1, wherein the alternating magnetic field shifts with a frequency in the range 10 kHz to 100 MHz and that the field strength in said coils amounts to min 0.1 and max 1000 Örstedt.

6. A device as claimed in claim 1, wherein said. magnetic field generating means are arranged in the form of a matrix so that a standard microtiter plate with 48 or 96 wells can constitute a sample container.

7. A device as claimed in claim 1, wherein said device comprises an automatic robotic sample handling system.

8. A device as claimed in claim 1, wherein the membrane-enveloped biological material is selected among stem cells, mammalian cells, malignant cells, plant cells, nerve cells, bacteria, viruses, cellular organelles, cell-membrane-enveloped structures, cell-wall-enveloped structures, liposomes, protozoa, parasites, and combinations thereof.

9. A device as claimed in claim 1, wherein the magnetically susceptible particles comprise a core of metal oxide and an envelope containing Concanavalin A, lectins, cell-binding proteins, cell-binding peptides, RNA, DNA, antibodies or genes.

10. A method for insertion of molecular units in multiple samples containing membrane-enveloped biological material and magnetically susceptible particles simnultaneously or sequentially, in which
d) each sample is inserted in a spatially limited area located in or in the vicinity of a magnetic field generating means;
e) the magnetic field generating means generates an alternating magnetic field by a computer program which controls the magnetic field generating means with respect to point of time and duration for activating each individual means, and
f) the molecular units are inserted in the membrane-enveloped biological material through the pores that are produced by the generated alternating magnetic field.

11. A method as claimed in claim 10, wherein the molecular units are selected among DNA, RNA, genes, proteins, antibodies, peptides and synthetic molecules.

12. A method as claimed in claim 10, wherein. the membrane-enveloped biological material is selected among stem cells, mammalian cells, malignant cells, plant cells, nerve cells, bacteria, viruses, cellular organelles, cell membrane-enveloped structures, cell-wall-enveloped structures, liposomes, protozoa, parasites, and combinations thereof.

13. A method as claimed in claim 10, wherein the magnetically susceptible particles comprise a core of a metal oxide and an envelope containing Concanavalin A, lectins, cell-binding proteins, cell-binding peptides, PKA, DNA, antibodies or genes.

14. A method for membrane passage in membrane-enveloped structures, gene transfer or transfection comprising utilizing the device of claim 1.

15. A method for insertion of molecular units in membrane-enveloped biological material selected among stem cells, mammalian cells, malignant cells, plant cells, nerve cells, bacteria, viruses, cellular organelles, cell-membrane-enveloped structures, cell-wall-enveloped structures, liposomes, protozoa, parasites and combinations thereof, comprising utilizing the device of claim 1.

16. A method claim as claimed in claim 15, wherein the molecular units are selected among DNA, RNA, genes, proteins, antibodies, peptides and synthetic molecules.

17. A robotic sample handling system or automatic sample preparation system for gene transfer comprising the device of claim 1.

18. A device as claimed in claim 2, wherein the magnetic field generating means are supplied with alternating currents of the same frequency and amplitude, and that said means are simultaneously supplied with said current.

19. A device as claimed in claim 2, wherein said device is equipped with a thermostat for accurate control of the temperature of said samples and/or that it is equipped with a variable timing for accurate control of the time during which said samples are exposed to the alternating magnetic field.

20. A device as claimed in claim 3, wherein said device is equipped with a thermostat for accurate control of the temperature of said samples and/or that it is equipped with a variable timing for accurate control of the time during which said samples are exposed to the alternating magnetic field.

21. A method as claimed in claim 11, wherein the magnetically susceptible particles comprise a core of a metal oxide and an envelope containing Concanavalin A, lectins, cell-binding proteins, cell-binding peptides, PKA, DNA, antibodies or genes.

22. A method as claimed in claim 12, wherein the magnetically susceptible particles comprise a core of a metal oxide and an envelope containing Concanavalin A, lectins, cell-binding proteins, cell-binding peptides, PKA, DNA, antibodies or genes.
